(19) United States
(12) Patent Application Publication
(10) Pub. No.: US 2004/0093011 A1
(43) Pub. Date: May 13, 2004

(54) Embolic protection device with lesion length assessment markers

(75) Inventor: Anthony C. Vrba, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/261,802

(22) Filed: Oct. 1, 2002

(51) Int. Cl. A61M 29/00

(52) U.S. Cl. 606/200

(57) ABSTRACT

An embolic protection filter assembly and method of assessing the size of an intravascular lesion. The filter assembly may include an elongate sheath having one or more marker members coupled thereto. The assembly may also include an elongate shaft having a filter coupled thereto.

### FIELD OF THE INVENTION

The present invention pertains to the field of embolic protection. More particularly, the present invention pertains to embolic protection filter assemblies that include one or more marker members, and methods of assessing the size of an intravascular lesion.

### BACKGROUND

Heart and vascular disease are majors problem in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion maybe mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

### BRIEF SUMMARY

The present invention pertains to embolic protection filter assemblies. In at least some embodiments, an embolic protection filter assembly may include an elongate shaft having an embolic protection filter coupled thereto. A delivery or retrieval sheath may be disposed at least partially over the shaft. One or more marker members may be coupled to the sheath. The marker members may allow a clinician to visualize the sheath and/or the assembly. Moreover, the marker members may allow a clinician to assess the size of an intravascular lesion.

### BRIEF DESCRIPTION OF THE DRAWINGS

FIG. 1 is an overview of an embolic protection filter assembly disposed within a blood vessel;

FIG. 2 is a side view of a first tubular member with a plurality of marker members disposed thereon; and

FIG. 3 is a side view of a second tubular member disposed over a first tubular member and a plurality of marker members.

### DETAILED DESCRIPTION

The following description should be read with reference to the drawings wherein like reference numerals indicate like elements throughout the several views. The detailed description and drawings illustrate example embodiments of the claimed invention.

When a clinician performs an intravascular intervention such as angioplasty, atherectomy, and the like, embolic debris may dislodge from the blood vessel that can travel in the bloodstream and impair blood flow, possibly leading to tissue damage. A number of intravascular filtering devices have been developed that can be disposed in the blood vessel downstream of the treatment site and expanded to capture debris. The present invention pertains to refinements of both embolic protection filter assemblies and methods of using them to measure the size of an intravascular lesion.

The size of the intravascular lesion very often dictates the particular diagnostic or therapeutic medical device that will be used to treat the lesion. For example, a relatively long lesion may need to be treated with a different (i.e., longer) angioplasty balloon or stent than a relatively short lesion. If an incorrectly sized device is used, it may not completely treat the target lesion. Therefore, a clinician may need to retract an incorrectly sized catheter and then advance a different one to the lesion. This process may repeatedly continue until the correctly sized medical device is located and positioned adjacent the lesion. Because of the variability in lesion size, is may be desirable to determine the size of the lesion before advancing the diagnostic or treatment catheter so that the correctly sized device can be used the first time. This, will allow the target lesion to be treated as effectively as possible and reduce amount of time and the costs associated (i.e., costs of using multiple devices) with repeatedly advancing different catheters to the lesion.

FIG. 1 is an overview of an example embolic protection filter assembly 10 disposed within a blood vessel 12. Assembly 10 includes a sheath 14 having one or more marker members 16. Marker members 16 can be used, for example, to measure the size of an intravascular lesion 18 as well as aid in the imaging of assembly 10 by comparing images of lesion with images of marker members 16. Assembly 10 may also include an elongate shaft or guidewire 20 having an embolic protection filter 22 coupled thereto.

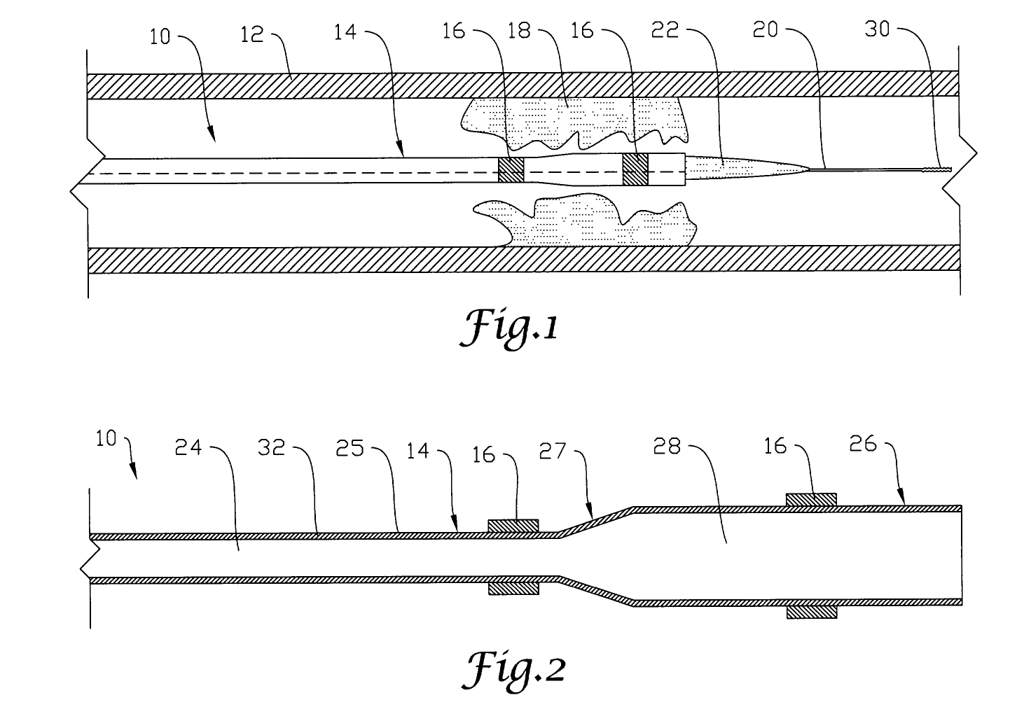

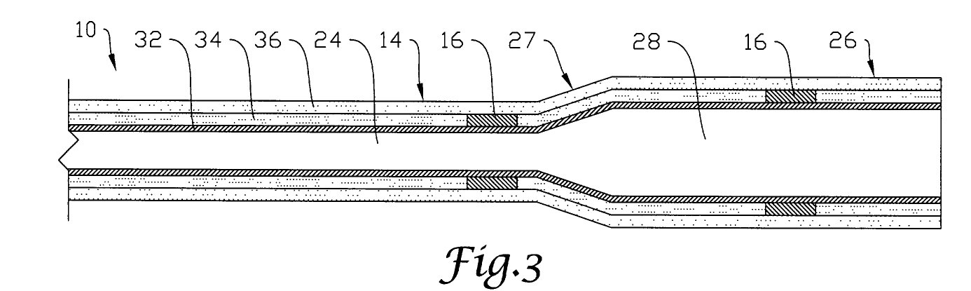

In at least some embodiments, sheath 14 is an embolic protection filter delivery sheath that is used to aid the delivery of filter 22 to an appropriate location adjacent lesion 18. According to this embodiment, sheath 14 is generally tubular and has a lumen 24 (as best seen in FIGS. 2 and 3) extending at least partially therethrough. Sheath 14 may include a proximal portion 25 and an enlarged or flared distal portion 26 separated by a necked portion 27. Distal portion 26 may define a filter holding chamber 28 (as best seen in FIGS. 2 and 3) that is configured for holding filter 22. Although sheath 14 is described as being a delivery sheath, it can be appreciated that sheath 14 may also be used to aid in the retrieval of filter 22. According to this embodiment, sheath 14 may comprise a retrieval sheath or a combination delivery-retrieval sheath.

Sheath 14 may be comprised of a metal, a polymer, a metal-polymer composite, or any other appropriate material. Some examples of suitable metals and metal alloys include stainless steel, such as 304 v stainless steel; nickel-titanium alloys such as super elastic or linear elastic nitinol, nickel-chromium alloy, nickel-chromium-iron alloy, cobalt alloy, or the like; or other suitable material. Some examples of suitable polymers include polyurethane, polyether-ester (for example a polyether-ester elastomer such as ARNITEL® available from DSM Engineering Plastics), polyester (for example a polyester elastomer such as HYTREL® available from DuPont), or linear low density polyethylene (for example REXELL®), polyamide (for example, DURETHAN® available from Bayer or CRISTAMID® available from Elf Atochem), elastomeric polyamides, block polyamide/ethers, polyether block amide (PEBA, for example available under the trade name PEBAX®), silicones, polyethylene, Marlex high-density polyethylene, polyetheretherketone (PEEK), polyimide (PI), and polyetherimide (PEI), a liquid crystal polymer (LCP) alone or blended with other materials, and the like, or mixtures, combinations, or copolymers thereof. The construction and composition of sheath 14 is described in greater detail below.

Marker members 16 may include a structure or combination of structures that are comprised of, coated with, or otherwise coupled to a radiopaque material. Radiopaque materials are understood to generally produce a relatively bright image on a fluoroscopy screen during a medical procedure. This relatively bright image aids the user of assembly 10 in determining its location. Radiopaque materials can include, but are not limited to, bismuth subcarbonate, iodine, gold, platinum, palladium, tantalum, tungsten or tungsten alloy, and the like. Some examples of suitable marker member 16 structures include a marker band of radiopaque material, a radiopaque coil, a polymeric material doped with a radiopaque material, and the like.

Marker members 16 may be disposed at essentially any location along the length of sheath 14. For example, a first marker member (indicated by reference number 16′) may be disposed adjacent distal portion 26 and a second marker member (indicated by reference number 16″) a distance proximal of first marker member 16′ (i.e., adjacent proximal portion 25). It can be appreciated, however, that alterations of the location of marker members 16 are within the scope of the invention. For example, more than one marker member 16 may be disposed adjacent proximal portion 25, distal portion 26, and/or necked portion 27. It can also be appreciated that alterations in the number of marker members 16 are also within the scope of the invention. For example, sheath 14 may include essentially any appropriate number of marker members 16 including three or more.

Marker members 16 may have a length (along the longitudinal axis of sheath 14) in the range of about 1 to 5 millimeters or longer. The length, by virtue of being known by the clinician, may be adapted and configured to allow the clinician to assess the size of lesion 18. For example, the clinician may use an appropriate imaging technique to generate an image of lesion 18 and marker members 16 and then compare the relative sizes thereof to estimate the size of lesion 18.

Similarly, marker members 16 may also be spaced or otherwise arranged to aid in the assessment of the size of lesion 18. For example, marker members 16 may be spaced a distance in the range of about 6 to 15 millimeters apart or further. Thus, the known spacing of marker members 16 (in addition to or independently of the known length of marker members 16) may be utilized when viewing images of lesion 18 generated by the imaging technique to estimate the size of lesion 18.

In at least some embodiments, assembly 10 may be configured as shown in FIG. 1 and advanced through blood vessel 12 toward lesion 18. It may be desirable to position filter 22 distal or “downstream” of lesion 18. According to this embodiment, assembly 10 is advanced across lesion 18 so that filter 22 can be appropriated placed. Images may be generated as assembly 10 crosses or is in the process of crossing lesion 18 so that the length and/or spacing of marker members 16 can be utilized to assess the size or length of lesion 18. When the size of lesion 18 is determined, sheath 14 may be retracted (leaving in place shaft 20 and filter 22) and an appropriately sized diagnostic or therapeutic medical device may be advanced over shaft 20 to a location adjacent lesion 18.

As described above, knowing the size of lesion 18 allows the clinician to utilize the most appropriately sized medical device for the desired intervention. This may desirable so that the entire lesion 18 or the intended portion of lesion 18 can be targeted and reached by the appropriately sized medical device. For example, it may be desirable to treat lesion 18 with an angioplasty catheter and/or a stent. According to this embodiment, it may be desirable to assess the size of lesion 18 so that the stent used can span the length of lesion 18. As stated above, knowing the size of lesion 18 can, thus, decrease the time necessary to perform the intervention, decrease the cost of the intervention by decreasing the number of devices used, as well as other advantages known to those of ordinary skill in the art.

As suggested above, shaft 20 may comprise an elongate medical device such as a guidewire, catheter (guide, diagnostic, or therapeutic), endoscopic device, arthroscopic device, etc. Shaft 20 can be made of any material suitable including metals, metal alloys, polymers, or the like, or combinations or mixtures thereof including those described above. The entire shaft 20 can be made of the same material, or in some embodiments, can include portions or sections made of different materials. In some embodiments, the material used to construct shaft 20 is chosen to impart varying flexibility and stiffness characteristics to different portions of shaft 20. For example, the material composition adjacent the proximal end of shaft 20 may be relatively stiff for pushability and torqueability, and the material composition adjacent the distal end of shaft 20 may be relatively flexible by comparison for better lateral trackability and steerability. Relatively stiff materials, for example, may include straightened 304v stainless steel wire, and relatively flexible materials may include, for example, a straightened super elastic or linear elastic alloy (e.g., nickel-titanium) wire. In addition, shaft 20 may generally taper near the distal end and may include a distal tip 30. Distal tip 30 may comprise a “spring tip” or “floppy tip” similar to analogous tips known in the art that helps make the distal end of shaft 20 be generally atraumatic to blood vessel walls, body organs, and tissue when advancing apparatus 10 through the vasculature.

Filter 22 may be disposed near the distal end of shaft 20. Filter 22 may generally comprise a number of configurations known to those skilled in the appropriate art. Filter 22 may include a filter frame, a filter material disposed over the frame, and one or more struts extending between the frame and shaft 20. In general, filter 22 operates between a first generally collapsed configuration and a second generally expanded configuration for collecting debris in a body lumen. The frame may be comprised of a “self-expanding” shape-memory material such as nickel-titanium alloy (to bias filter 22 to be in the second expanded configuration). The filter material may be comprised of a polyurethane sheet and include at least one opening that may be, for example, formed by known laser techniques. The holes or openings are sized to allow blood flow therethrough but restrict flow of debris or emboli floating in the body lumen or cavity.

Sheath 14 may be comprised of a single layer of material with marker members 16 coupled thereto. Alternatively, sheath 14 may be comprised of a plurality of layers. According to this embodiment, sheath 14 may include a first generally tubular member 32 having marker members 16 coupled thereto as illustrated in FIG. 2. First member 32 may be comprised of any appropriate material including those listed herein. For example, first member 32 may be comprised of a polymer that is extruded, molded (e.g., over a mandrel), or otherwise formed to have the desired shape.

Additionally, in some embodiments, a lubricious (e.g., hydrophylic) or other type of coating may be applied over portions or all of first member 32. Hydrophobic coatings such as fluoropolymers, including polytetrafluroethylene (PTFE), provide a dry lubricity that may, for example, allow first member 32 to more easily separate from a manufacturing mandrel, allow shaft 20 to slide more freely therein, etc. Suitable lubricious polymers are well known in the art and may include hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

Manufacturing of sheath 14 may include disposing marker members 16 over first member 32. Marker members 16 may be coupled to sheath 14 in a number of ways. For example, marker members 16 may be adhesively bonded, heat bonded, friction fit, or otherwise disposed adjacent first member 32.

A second generally tubular member 34 may be disposed over first member 32 and marker members 16 as illustrated in FIG. 3. In at least some embodiments, second member 34 may be comprised of a heat shrink tube that can be disposed over first member 32 and marker members 16. According to this embodiment, second member 34 may aid in the coupling of marker members 16 to first member 32. It can be appreciated that second member 32 may be comprised of other materials including those listed above and may or may not be comprised of the same material as first member 32.

After disposing second member 34 over first member 32, sheath 14 may be ground or otherwise manufactured so that the outer surface thereof is generally smooth. In some embodiments, sheath 14 may be smoothed by disposing a third member 36 over second member 34. Similar to what is described above, third member 36 may be comprised of any of the material listed herein and may be useful for aiding the process of smoothing the outer surface of sheath 14.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An embolic protection filter assembly, comprising:
an elongate sheath having a proximal end, a distal end, and a lumen extending at least in part therethrough;
a first radiopaque marker member coupled to the sheath adjacent the distal end;
an elongate shaft disposed within the lumen, the shaft including a proximal end and a distal end; and
a filter on the shaft adjacent the distal end of the shaft.

2. The embolic protection filter assembly of claim 1, wherein the sheath includes a necked region and an enlarged distal region.

3. The embolic protection filter assembly of claim 1, wherein the first marker member is coupled to the sheath at the distal region.

4. The embolic protection filter assembly of claim 1, wherein the first marker member has a length of about one to five millimeters.

5. The embolic protection filter assembly of claim 1, further comprising a second marker member coupled to the sheath proximally of the first marker member.

6. The embolic protection filter assembly of claim 5, wherein the first marker member and the second marker member are spaced about 6 to 15 millimeters apart.

7. An embolic protection filter assembly, comprising:
a delivery sheath having a proximal region, a necked region, an enlarged distal region, and a lumen extending therethrough;
a guidewire disposed within the lumen, the guidewire including a distal end;
a filter coupled to the guidewire adjacent the distal end thereof; and
means for measuring a lesion coupled to the sheath.

8. The embolic protection filter assembly of claim 7, wherein means for measuring a lesion include one or more radiopaque markers coupled to the sheath.

9. A method of measuring the size of an intravascular lesion, comprising the steps of:
providing an embolic protection filter assembly including an elongate sheath having a lumen, an elongate shaft disposed within the lumen, and a filter coupled to the shaft, wherein the sheath includes one or more marker members;
disposing a contrast agent adjacent an intravascular lesion to visualize the lesion;
advancing the assembly through a blood vessel to a location adjacent the lesion;
generating an image of the one or more marker members and the lesion with one or more imaging devices; and
measuring the size of the lesion by comparing the image of the one or more marker members with the image of the lesion.

10. The method of claim 9, wherein the filter is collapsed within the sheath and biased to be in an expanded configuration, and further comprising the step of retracting the sheath relative to the shaft so that the filter shifts to the expanded configuration.

11. The method of claim 10, further comprising the step of advancing a therapeutic device over the shaft to a location adjacent the lesion.

12. The method of claim 11, further comprising the step of using the filter to filter embolic debris generated as a result of using the therapeutic device to treat the lesion.

13. An embolic protection filter assembly, comprising:
an inner tube having a proximal end, a distal end, and a lumen extending therethrough;
one or more marker members coupled to the inner tube adjacent the distal end;
an outer sheath disposed over the inner tube and the one or more marker members;
a guidewire disposed within the lumen of the inner tube, the guidewire including a distal end; and
a filter coupled to the guidewire adjacent the distal end thereof.

14. The embolic protection filter assembly of claim 13, wherein the inner tube includes a necked region and an enlarged distal region.

15. The embolic protection filter assembly of claim 14, wherein at least one of the one or more marker members are coupled to the inner tube at the distal region.

16. The embolic protection filter assembly of claim 13, wherein the one or more marker members have a length of about one to five millimeters.

17. The embolic protection filter assembly of claim 13, wherein the assembly includes at least two marker members and the two marker members are spaced about 6 to 15 millimeters apart.

18. An embolic protection filter assembly, comprising:
a filter delivery sheath having a proximal region, a distal region, and a lumen extending at least partially therethrough;
a guidewire disposed within the lumen;
a filter coupled to the guidewire;
wherein at least a portion of the filter is collapsed and disposed within the sheath;
a first marker member coupled to the distal region of the sheath;
a second marker member coupled to the distal region of the sheath; and
wherein the first marker member and the second marker member are adapted and configured to permit a clinician to measure the size of an intravascular lesion by visualizing the marker members and the lesion with one or more imaging devices.

19. The embolic protection filter assembly of claim 18, wherein the first marker member has a length of about 1 to 5 millimeters.

20. The embolic protection filter assembly of claim 18, wherein the first marker member and the second marker member are spaced about 6 to 15 millimeters apart.
